(19) United States
(12) Patent Application Publication
(10) Pub. No.: US 2021/0283414 A1
(43) Pub. Date: Sep. 16, 2021

(54) MICROWAVE TREATMENT OF SKIN

(71) Applicant: Emblation Ltd, Alloa (GB)

(72) Inventors: Gary Beale, Alloa (GB); Eamon McErlean, Alloa (GB); Shailesh Joshi, Alloa (GB); Matthew Kidd, Alloa (GB)

(21) Appl. No.: 17/202,876

(22) Filed: Mar. 16, 2021

Related U.S. Application Data
(60) Provisional application No. 62/989,957, filed on Mar. 16, 2020.

(51) Int. Cl. A61N 5/02

(52) U.S. Cl. CPC A61N 5/022

(57) ABSTRACT

The present disclosure provides microwave-based methods for the modulation of certain genes and immunomodulatory factors. The various methods described herein may be used to modulate the expression of one or more genes thought to be beneficial in and/or associated with, the treatment and/or prevention of a disease and/or condition of the skin.

### CROSS-REFERENCE TO RELATED APPLICATIONS

This application claims the benefit of the filing dates of U.S. Provisional Patent Application No. 62/989,957, filed on Mar. 16, 2020, the entire contents of which is incorporated herein by reference.

### FIELD

The present disclosure provides microwave-based methods for the modulation of certain genes and immunomodulatory factors.

### BACKGROUND

In most energy-based treatment systems, such as electromagnetic (EM) ablation systems using microwaves, electromagnetic radiation is delivered from a generator, via a connecting cable, to an energy delivering applicator placed in or onto tissue.

It is possible to treat skin conditions by modulating a patient's immune system. Commonly used methods are based on topical, therapeutic and pharmacological means. Topical immunomodulators comprise both immunostimulatory and immunosuppressive agents and may cause or induce a cytokine secretion.

Imidazoquinolines such as Imiquimod can activate monocytes, macrophages and dendritic cells by binding to Toll-like receptor 7 and 8 (TLR-7, TLR-8) on the cell surface causing NFkB-dependent release of proinflammatory cytokines such as IFNα, TNF-α, and IL-12 and chemokines like IL1, IL6, IL8, and IL10. Imiquimod is used in the treatment of several skin conditions such as warts, basal cell carcinoma (BCC), molluscum contagiosum, melanoma metastases and other pre-cancerous and cancerous lesions such as actinic keratoses, Bowen's disease, cutaneous T-cell lymphoma etc.

Other commonly used topical agents include 5-Fluorouracil (5-FU), Diclofenac gel and Ingenol [1] [2].

Topical corticosteroids are also used as immunosuppressive agents but are known to cause long term suppressive effects on the connective tissue, seen as skin atrophy or resistance to therapy [3] [4].

Therapeutic techniques in treating skin conditions by immunomodulation are less common than topical methods. Photodynamic therapy (PDT) when used in conjunction with topical 5-aminolaevulinic acid (ALA) (also called as ALA-PDT) has shown promising results in the treatment of viral warts, actinic keratosis, superficial basal cell carcinomas and Bowen's disease [5] [1]. Other phototherapeutic modalities such as polarized light therapy (PLT), UV-A and UV-B therapies, low level laser therapy (LLLT), light emitting diode (LED) therapy and infrared (IR) therapy have also shown both inflammatory and anti-inflammatory effects [6].

Gene therapy may be used to achieve targeted gene expression. Using moderate hyperthermia (prolonged exposure to temperature 39° C. to 43° C.), expression of a heterologous gene with a heat shock protein 70 (HSP 70) promoter was shown to be elevated 500-1000 fold along with increased TNF and cytokine signalling [7].

Local hyperthermia induced by far-infrared has been effective in treating HPV related skin conditions such as condyloma acuminatum (also known as anogenital warts) where immunomodulatory effects such as increased levels of CD1a+/CD83+LCs and decreased levels of CCR6 mRNA were observed indicating migrational maturation of Langerhans cells (LCs) [8].

While these topical, therapeutic and pharmacological therapies have shown promising results, they are aggressive, take longer to be effective and often lead to adverse side effects such as significant local inflammation, dermal ulcer, burning sensation, skin rash, flaking, swelling, desquamation, edema, excoriation, exfoliation of skin, pruritus, skin erosion, erythema, breathing difficulties, allergic rhinitis, depigmentation, scarring and high recurrence rates [10] [11] [13].

Corr et al have provided a method for treating solid tumours, comprising a combination of radiofrequency therapy (RF) and immunotherapy where immunotherapy utilises additional immune checkpoint inhibitors, therapeutic vaccines and other drugs that influence immune cell function to enhance anti-cancer activity [15].

Further, combinational treatments such as pharmaceutical composition for hsp90 inhibitors to enhance tumour immunogenicity and chemokine based therapy analysed using differential gene expression have been proposed [16] [17].

The effects of chemotherapy agents such as Gemcitabine on breast cancer have been studied using differential gene expression analysis [18]. Radvanyi et. al. have shown adoptive cell therapy used as an immunotherapy for metastatic melanoma and analysed it using similar approach [19]. Equivalent methods have been applied in understanding proinflammatory gene expression in treating sinus rhythm and atrial fibrillation [20].

All these methods are either pharmacological, posing higher side effects or are used in combination with other immunomodulatory agents. Accordingly, there is a need for a standalone technology to provide a therapeutic level of immunomodulation via the modulation of gene expression. Such a technology may find application in the treatment of a variety of skin diseases/conditions. The present invention addresses that need.

### SUMMARY

In a first aspect, there is provided a microwave system for use in a method of modulating the expression of one or more genes.

The disclosure further provides microwave energy for use in a method of modulating the expression of one or more genes.

There is also provided a method of modulating the expression of one or more genes, said method comprising administering microwave energy to a subject in need thereof.

The microwave energy may be supplied by a microwave generator and administered to a subject at a frequency of between about 900 MHz and about 200 GHz. By way of example, the microwave energy may be administered (via a microwave energy generator) at about 915 MHz, at about 2.45 GHz, at about 5.8 GHz, at about 8.0 GHz, or at about 24.125 GHz.

Microwave energy for use in the various methods described herein can comprise an input power of 0.5 W to 40 W. The input power may be applied for a duration of anywhere between about 0.1 s to 20 s. A higher power may be paired with a brief (dose) duration; a lower power may be paired with longer (dose) duration. For example a useful dose of microwave energy may comprise 5 W administered for 3s, 4 W administered for 3s or 3 W administered for 3s.

In some embodiments, the microwave energy may be administered as a series of pulses. A pulsed administration may comprise, for example, the use of one continuous energy discharge “pulse envelope or dose” with a gap between each subsequent “dose”.

The “pulse envelope or dose” may contain or comprise continuous wave or pulse modulated energy e.g. (1 kHz modulation). The microwave energy may be administered as a series of pulses with a time gap of anywhere between about 1 s to about 60 s between each pulse and/or single energy administration. For example a pulsed treatment may be repeated 3 times with a 20 s-time gap in between each single administration of energy.

The microwave treatment maybe comprise microwave energy which is ‘non-ablative’, ‘mildly ablative’, or ablative. A ‘non-ablative’ treatment may comprise only a treatment duration —perhaps, for example a treatment duration of about 1-2s or more. A ‘non-ablative’ treatment might comprise the use of microwave energy at a very low energy level energy, so as to cause no direct tissue or skin damage. Without wishing to be bound by theory, a ‘non-ablative’ treatment may use or exploit non-thermal mechanisms (high electric fields, interruption or modulation of intra-cellular signalling/ion channels).

A ‘mildly ablative’ treatment with microwave energy may comprise a treatment duration of about 2-5s or more. The total amount of energy used may be low so as to cause no direct damage and only a mild to moderate elevation of temperature. A mildly-ablative treatment may produce modest thermal effects (heat shock elevation, mild inflammation etc.) and promote apoptosis within (or of) treated tissue.

An ‘ablative’ treatment comprises the use of a moderate to higher level of microwave energy. The microwave energy may be used for a prolonged duration of around 5-10s or more. This may result in some direct tissue damage, a moderate to high level of temperature elevation (within the treated tissue) and potentially some direct tissue damage/necrosis.

A useful microwave-based treatment may be repeated any required number of times. A treatment may be repeated any suitable or required number of times between about 1 and 6 times for example 2, 3, 4 or 5 times. The interval between each treatment may comprise anywhere between about 1 and about 6 weeks, for example, about 2 weeks, about 3 weeks, about 4 weeks or about 5 weeks.

It should be noted that the specifics of a useful dose may vary depending on the gene(s) to be modulated, the subject (age, weight, condition, history etc.) and the disease or condition to be treated and/or prevented. One of skill will be able to tweak any aspect of the microwave energy dose to fit the clinical circumstances.

The various methods described herein may be applied to subjects in need of treatment, wherein a subject in need of treatment is a human or animal subject suffering from and/or predisposed to a disease or condition of the skin (see below for a list of specific diseases and/or conditions).

Moreover, the described methods may be applied to animals and humans in-situ, in-vivo and ex-vivo.

The methods may be applied to biopsies, samples (provided by or obtained from a subject) and in vitro. Accordingly, the disclosure provides an in vitro method of modulating the expression of one or more genes, said method comprising exposing a tissue to microwave energy.

The disclosure also provides a method of treating or preventing a skin condition by modulating the expression of one or more genes, said method comprising exposing a subject suffering from, or predisposed to, the skin condition, to microwave energy. Such methods may be applied to a diseased or affected tissue in the subject to be treated.

The microwave system may comprise a microwave generator; a controller configured to control the microwave generator to generate microwave energy having a selected operational frequency or range of frequencies; a microwave cable configured to deliver the microwave energy to a microwave antenna extending from or coupled to a distal end of the microwave cable; and the microwave antenna.

The disclosure is based on the finding that when applied to a tissue, microwave energy is able to modulate gene expression. Moreover, the invention is further based on the finding that microwave energy administered at any of the described doses, may be used to modulate gene expression.

In some cases the expression of certain genes is downregulated (inhibited or reduced). That is to say, when compared to the expression of those same genes in a tissue which has not been exposed to microwave energy, there is a lower level of expression of those genes in the exposed tissue.

In other cases the expression of certain genes is upregulated (induced, promoted or stimulated). That is to say, when compared to the expression of those same genes in a tissue which has not been exposed to microwave energy, there is a higher level of expression of those genes in the exposed tissue.

A microwave system of the type described above may be used to expose any given tissue to microwave energy. The tissue may be a biological tissue.

A tissue to be exposed to microwave energy (for the purpose of modulating the expression of one or more genes within that tissue) may comprise diseased tissue. A diseased tissue may be any tissue exhibiting the signs or symptoms characteristic of one or more diseases.

The tissue may comprise skin.

The tissue may also comprise diseased skin. Diseased skin may exhibit the signs or symptoms characteristic of one or more diseases and/or conditions associated with the skin. Skin which may benefit from treatment using microwave energy may include aging skin and/or skin which exhibits solar damage and/or the signs and/or symptoms associated therewith. Microwave energy may also be applied to the skin with one or more scars, erosion and/or lesions.

Without wishing to be bound by theory, it is suggested that following exposure to microwave energy, one or more genes within skin (as defined above) may be modulated such that some aspect of a disease (for example one or more symptoms) or the appearance and/or texture of the skin is improved or resolved. In other words, microwave energy may be used to modulate the expression of one or more genes so as to have a beneficial effect up a symptom or characteristic of the various skin related diseases and/or conditions noted above.

The tissue may belong or be derived, provided or obtained by/from a subject to be treated using a method described herein. Accordingly, the tissue may be an in-situ tissue, in vivo tissue or a biopsy of ex vivo sample.

As stated, a subject to be treated using a method described herein may be suffering from or predisposed/susceptible to, one or more conditions. For example, a subject to be treated may be suffering from or predisposed/susceptible to, one or more diseases of the skin and/or cancer.

Accordingly, and by way of example, a method described herein may be applied to the skin of a subject. The subject may be suffering from one or more diseases of the skin.

The various methods described herein may be applied so as to modulate the expression of one or more genes thought to be beneficial in the treatment and/or prevention of a disease and/or condition of the skin—including any of the specific diseases and/or conditions described herein.

A subject to be treated may be suffering from one or more diseases of the skin, including, but not limited to warts, eczema, psoriasis, acne, cherry angioma, hidradenitis suppurativa, rosacea, ichthyosis, keloid scars, seborrheic dermatitis, seborrheic keratosis, seborrheic hyperplasia, Sebaceous hyperplasia, basal cell carcinoma, actinic keratosis, syringoma, squamous cell carcinoma, nevus, lentigo maligna, Melasma, melanoma, milia, molluscum contagiosum, cervical intraepithelial neoplasia, vaginal intraepithelial neoplasia, vulvar intraepithelial neoplasia, Bowen's disease and/or erythroplasia of queyrat. A method of this disclosure may be applied to the treatment or prevention of any of these diseases.

The methods of this disclosure may also be applied to the treatment of diseases or conditions such as gastric epithelial dysplasia, cardiovascular lesions, conditions involving oral cavity such as epithelial dysplasia, leukoplakia, hairy leukoplakia, erythroplakia, erythroleukoplakia, lichen planus, xerostomia, mucositis, pyogenic granuloma, angioma, nicotinic stomatitis, actinic cheilitis, keratoacantoma, hyperkeratosis, candidosis, erythema migrans and/or canker sores.

Any modulation of gene expression may be determined by rRNA (ribosomal RNA), tRNA (transfer RNA), and ncRNAs (noncoding RNA) or mRNA (messenger RNA) transcript analysis. An analysis of this type may be described as generating a “transcriptome”. By of example, the transcriptome of a tissue exposed to microwave energy (a test transcriptome) may be compared to the transcriptome of a tissue which has not been exposed to microwave energy (a control transcriptome). Any effect of microwave energy on a level of gene expression will manifest as a difference in the level of expression between the test and the control transcriptomes. A transcriptome may be generated using any suitable technique.

Suitable techniques include, but are not limited to real-time quantitative PCR (qPCR), Microarrays or RNA-Seq (RNA-sequencing) A transcriptome analysis may be generated by earlier techniques for example ESTs (expressed sequence tags), northern blotting, nylon membrane arrays and SAGE (serial analysis of gene expression).

Without wishing to be bound by theory, the application of microwave energy to a tissue may induce hyperthermia within said tissue. That hyperthermia may be referred to as a “local hyperthermia” or “regional hyperthermia”. The application of microwave energy may, for example, release beneficial intracellular and/or extracellular HSPs (heat shock proteins), and also may induce non-thermal effects such as but not limited to, dielectrophoretic effects, electrophoresis effects, electroosmosis effects, electroporation effects, high frequency (GHz) mechanical resonance effects (relating to fracturing viral particles), stress causing enhancement of protein reaction rates, optimised immunomodulatory signalling, improved enzyme stability, improved cellular uptake and cellular function of cell, homogeneous orientation of large molecules. The sum total of all of these effects may be the modulation (i.e. the up- or down-regulation) of one or more of the genes described herein.

By way of example, the methods and uses described herein may be applied to the modulation of the expression of one or more of the genes.

The gene or genes to be modulated may be directly or indirectly associated with a disease or condition affecting the skin. For example, one or more of the genes may be involved or linked with/to one or more pathways or mechanisms associated with a disease or condition of the skin.

The gene or genes to be modulated may encode or provide factors associated with the host immune system. For example the gene or genes to be modulated may encode or provide factors which are immunomodulatory.

Additionally or alternatively, the gene or genes to be modulated may be classified as “cancer” or “oncogenic” genes—that is to say, their expression has been associated with one or more types of cancer.

In view of the above, one of skill will appreciate that where a disease or condition of the skin is known to be associated with a level of expression of a particular gene, microwave energy may represent a novel route to the treatment and/or prevention of that disease or condition. By way of example, microwave energy may be used to restore the aberrant expression of the one or more genes that is known to be associated with the disease or condition.

Where the downregulation or inhibition of a particular gene or genes is associated with a disease or condition of the skin (for example the downregulation of a particular gene or genes causes or contributes to a particular skin disease or condition), microwave energy may be used to upregulate those genes, restoring the level of expression, activity and/or function that is required for healthy skin.

Alternatively, where the upregulation of a particular gene or genes is associated with a disease or condition of the skin (for example the upregulation of a particular gene or genes causes or contributes to a particular skin disease or condition), microwave energy may be used to downregulate or suppress those genes so that an appropriate or normal level of expression is restored.

It should also be noted that where the upregulation of the expression of one or more gene(s) is associated with the treatment and/or prevention of a particular skin disease or condition, microwave energy may be used to affect the upregulation of those gene(s). Such microwave induced upregulation would be recognised as helping to treat and/or prevent the diseases and/or condition of the skin.

Alternatively, where downregulation (or inhibition) of the expression of one or more gene(s) is associated with the treatment and/or prevention of a particular skin disease or condition, microwave energy may be used to affect the downregulation and/or inhibition of those gene(s). Such microwave induced gene inhibition would be recognised ad helping to treat and/or prevent the diseases and/or condition of the skin.

Genes which can be modulated by the methods described herein (i.e. by the application of microwave energy) include one or more of those listed in Table 1. Specifically, microwave energy may be used to upregulate the expression of one or more of the genes listed in Table

TABLE 1

| Gene | Official full name | Immunomodulatory pathway participation |
|---|---|---|
| CFH | complement factor H | Complement System, |
| | | Host-pathogen Interaction |
| | | Innate Immune System |
| MSR1 | macrophage scavenger | Phagocytosis and Degradation |
| | receptor 1 | |
| CXCL12 | chemokine (C-X-C motif) ligand | Chemokine Signaling |
| | 12 | Cytokine Signaling |
| | | Lymphocyte Trafficking |
| | | NF-kB Signaling |
| HLA-DPB1 | major histocompatibility | Adaptive Immune System |
| | complex, class II, DP beta 1 | Cell Adhesion |
| | | Cytokine Signaling |
| | | Host-pathogen Interaction |
| | | Lymphocyte Activation |
| | | MHC Class II Antigen Presentation |
| | | Phagocytosis and Degradation |
| | | T Cell Receptor Signaling |
| | | Type II Interferon Signaling |
| MRC1 | mannose receptor, C type 1 | Adaptive Immune System |
| | | Host-pathogen Interaction |
| | | MHC Class I Antigen Presentation |
| | | Phagocytosis and Degradation |
| FCER1A | Fc fragment of IgE, high affinity | Innate Immune System |
| | I, receptor for; alpha | |
| | polypeptide | |
| C3 | complement component 3 | Adaptive Immune System |
| | | Complement System |
| | | Host-pathogen Interaction |
| | | Innate Immune System |
| | | Phagocytosis and Degradation |
| VCAM1 | vascular cell adhesion molecule | Adaptive Immune System |
| | 1 | Cell Adhesion |
| | | Cytokine Signaling |
| | | Host-pathogen Interaction |
| | | Lymphocyte Activation |
| | | Lymphocyte Trafficking |
| | | NF-kB Signaling |
| | | TNF Family Signaling |
| | | Type II Interferon Signaling |
| CFD | complement factor D (adipsin) | Complement System |
| | | Hemostasis |
| | | Host-pathogen Interaction |
| | | Innate Immune System |
| CCL13 | chemokine (C-C motif) ligand | Chemokine Signaling |
| | 13 | Cytokine Signaling |
| | | NF-kB Signaling |
| LGALS3 | lectin, galactoside-binding, | |
| | soluble, 3 | |
| CDH5 | cadherin 5, type 2 (vascular | Cell Adhesion |
| | endothelium) | Lymphocyte Trafficking |
| KIT | v-kit Hardy-Zuckerman 4 feline | Cytokine Signaling |
| | sarcoma viral oncogene | Lymphocyte Activation |
| | homolog | |
| CD209 | CD209 molecule | Adaptive Immune System |
| | | Host-pathogen Interaction |
| | | Innate Immune System |
| | | Lymphocyte Activation |
| | | Phagocytosis and Degradation |
| LILRB4 | leukocyte immunoglobulin-like | Adaptive Immune System |
| | receptor, subfamily B (with TM | |
| | and ITIM domains), member 4 | |
| CLEC5A | C-type lectin domain family 5, | Innate Immune System |
| | member A | |
| HLA-DRA | major histocompatibility | Adaptive Immune System |
| | complex, class II, DR alpha | Cell Adhesion |
| | | Cytokine Signaling |
| | | Host-pathogen Interaction |
| | | Lymphocyte Activation |
| | | MHC Class II Antigen Presentation |
| | | Phagocytosis and Degradation |
| | | T Cell Receptor Signaling |
| | | Type II Interferon Signaling |
| SERPING1 | serpin peptidase inhibitor, clade | Complement System |
| | G (C1 inhibitor), member 1 | Hemostasis |
| | | Host-pathogen Interaction |
| | | Innate Immune System |
| HLA-DPA1 | major histocompatibility | Adaptive Immune System |
| | complex, class II, DP alpha 1 | Cell Adhesion |
| | | Cytokine Signaling |
| | | Host-pathogen Interaction |
| | | Lymphocyte Activation |
| | | MHC Class II Antigen Presentation |
| | | Phagocytosis and Degradation |
| | | T Cell Receptor Signaling |
| | | Type II Interferon Signaling |
| CD4 | CD4 molecule | Adaptive Immune System |
| | | Cell Adhesion |
| | | Cytokine Signaling |
| | | Innate Immune System |
| | | Lymphocyte Activation |
| | | T Cell Receptor Signaling |
| FCGRT | Fc fragment of IgG, receptor, | Hemostasis |
| | transporter, alpha | |
| NT5E | 5′-nucleotidase, ecto (CD73) | Immunometabolism |
| C2 | complement component 2 | Complement System |
| | | Host-pathogen Interaction |
| | | Innate Immune System |
| CSF1 | colony stimulating factor 1 | Cytokine Signaling |
| | (macrophage) | TNF Family Signaling |
| HAVCR2 | hepatitis A virus cellular | Cytokine Signaling |
| | receptor 2 | Lymphocyte Activation |
| TNFSF12 | tumor necrosis factor (ligand) | Cytokine Signaling |
| | superfamily, member 12 | |
| LAIR1 | leukocyte-associated | Adaptive Immune System |
| | immunoglobulin-like receptor 1 | Innate Immune System |
| B2M | beta-2-microglobulin | Adaptive Immune System |
| | | Cytokine Signaling |
| | | Innate Immune System |
| | | Lymphocyte Activation |
| | | MHC Class I Antigen Presentation |
| | | Type II Interferon Signaling |
| CMKLR1 | chemokine-like receptor 1 | Immunometabolism |
| PDCD1LG2 | programmed cell death 1 ligand | Adaptive Immune System |
| | 2 | Cell Adhesion |
| | | Lymphocyte Activation |
| CD45R0 | protein tyrosine phosphatase, | Adaptive Immune System |
| | receptor type, C | Cell Adhesion |
| | | Innate Immune System |
| | | Lymphocyte Activation |
| | | T Cell Receptor Signaling |
| CD34 | CD34 molecule | Cell Adhesion |
| XCR1 | chemokine (C motif) receptor 1 | Chemokine Signaling |
| | | Cytokine Signaling |
| CYBB | cytochrome b-245, beta | Adaptive Immune System |
| | polypeptide | Host-pathogen Interaction |
| | | Innate Immune System |
| | | Lymphocyte Trafficking |
| | | MHC Class I Antigen Presentation |
| | | NLR signaling |
| | | Oxidative Stress |
| | | Phagocytosis and Degradation |
| ITGAM | integrin, alpha M (complement | Cell Adhesion |
| | component 3 receptor 3 | Complement System |
| | subunit) | Cytokine Signaling |
| | | Hemostasis |
| | | Host-pathogen Interaction |
| | | Innate Immune System |
| | | Lymphocyte Trafficking |
| | | Phagocytosis and Degradation |
| | | TLR Signaling |
| TGFBR2 | receptor II (70/80kDa) | Cytokine Signaling |
| | | Host-pathogen Interaction |
| | | transforming growth factor, beta |
| | | Lymphocyte Activation |
| | | TGF-b Signaling |
| | | Th17 Differentiation |
| | | Treg Differentiation |
| HLA-DMB | major histocompatibility | Adaptive Immune System |
| | complex, class II, DM beta | Cell Adhesion |
| | | Host-pathogen Interaction |
| | | Lymphocyte Activation |
| | | MHC Class II Antigen Presentation |
| | | Phagocytosis and Degradation |
| CTSS | cathepsin S | Adaptive Immune System |
| | | Apoptosis |
| | | Host-pathogen Interaction |
| | | Innate Immune System |
| | | MHC Class I Antigen Presentation |
| | | MHC Class II Antigen Presentation |
| | | Phagocytosis and Degradation |
| | | TLR Signaling |
| IKZF2 | IKAROS family zinc finger 2 | Transcriptional Regulation |
| | (Helios) | |
| CD59 | CD59 molecule, complement | Complement System |
| | regulatory protein | Innate Immune System |
| | | Lymphocyte Activation |
| TNFRSF11A | tumor necrosis factor receptor | Cytokine Signaling |
| | superfamily, member 11a, | NF-kB Signaling |
| | NFKB activator | |
| TNFSF13B | tumor necrosis factor (ligand) | Cytokine Signaling |
| | superfamily, member 13b | Lymphocyte Activation |
| | | NF-kB Signaling |
| TLR3 | toll-like receptor 3 | Host-pathogen Interaction |
| | | Innate Immune System |
| | | TLR Signaling |
| STAT5A | signal transducer and activator | Cytokine Signaling |
| | of transcription 5A | Host-pathogen Interaction |
| | | Th2 Differentiation |
| | | Transcriptional Regulation |
| IFITM1 | interferon induced | Adaptive Immune System |
| | transmembrane protein 1 (9-27) | B cell Receptor Signaling |
| | | Cytokine Signaling |
| | | Type I Interferon Signaling |
| NFKB1 | nuclear factor of kappa light | Adaptive Immune System |
| | polypeptide gene enhancer in | Apoptosis |
| | B-cells 1 | B cell Receptor Signaling |
| | | Chemokine Signaling |
| | | Cytokine Signaling |
| | | Host-pathogen Interaction |
| | | Inflammasomes |
| | | Innate Immune System |
| | | NF-kB Signaling |
| | | NLR signaling |
| | | Oxidative Stress |
| | | T Cell Receptor Signaling |
| | | Thi Differentiation |
| | | TNF Family Signaling |
| | | TLR Signaling |
| | | Transcriptional Regulation |
| ITGB2 | integrin, beta 2 (complement | Adaptive Immune System |
| | component 3 receptor 3 and 4 | Cell Adhesion |
| | subunit) | Complement System |
| | | Cytokine Signaling |
| | | Hemostasis |
| | | Host-pathogen Interaction |
| | | Innate Immune System |
| | | Lymphocyte Activation |
| | | Lymphocyte Trafficking |
| | | Phagocytosis and Degradation |
| | | TLR Signaling |
| C1S | complement component 1, s | Complement System |
| | subcomponent | Host-pathogen Interaction |
| | | Innate Immune System |
| SDHA | succinate dehydrogenase | Cytokine Signaling |
| | complex, subunit A, | |
| | flavoprotein (Fp) | |
| ETS1 | v-ets erythroblastosis virus E26 | Host-pathogen Interaction |
| | oncogene homolog 1 (avian) | Oxidative Stress |
| CASP1 | caspase 1, apoptosis-related | Transcriptional Regulation |
| | cysteine peptidase (interleukin | Cytokine Signaling |
| | 1, beta, convertase) | Host-pathogen Interaction |
| | | Inflammasomes |
| | | Innate Immune System |
| | | NLR signaling |
| C1R | complement component 1, r | Complement System |
| | subcomponent | Host-pathogen Interaction |
| | | Innate Immune System |
| | | Phagocytosis and Degradation |
| HLA-DMA | major histocompatibility | Cell Adhesion |
| | complex, class II, DM alpha | Host-pathogen Interaction |
| | | MHC Class II Antigen Presentation |
| | | Phagocytosis and Degradation |
| CD74 | CD74 molecule, major | Adaptive Immune System |
| | histocompatibility complex, | Hemostasis |
| | class II invariant chain | Host-pathogen Interaction |
| | | Lymphocyte Activation |
| | | MHC Class II Antigen Presentation |
| MAPK1 | mitogen-activated protein | Apoptosis |
| | kinase 1 | Autophagy |
| | | B cell Receptor Signaling |
| | | Chemokine Signaling |
| | | Cytokine Signaling |
| | | Hemostasis |
| | | Host-pathogen Interaction |
| | | Innate Immune System |
| | | Lymphocyte Activation |
| | | NLR signaling |
| | | T Cell Receptor Signaling |
| | | TGF-b Signaling |
| | | TNF Family Signaling |
| | | TLR Signaling |
| IL6ST | interleukin 6 signal transducer | Cytokine Signaling |
| | (gp130, oncostatin M receptor) | Lymphocyte Activation |
| | | Th17 Differentiation |

The methods and uses described herein may also be applied to modulating the expression of one or more of the genes listed in Table 2. Microwave energy may be used to downregulate the expression of one or more of the genes listed in Table 2.

!TABLE 2

| Gene | Official full name | Immunomodulatory pathway participation |
|---|---|---|
| IL8 | interleukin 8 | Chemokine Signaling |
| | | Cytokine Signaling |
| | | Host-pathogen Interaction |
| | | NF-kB Signaling |
| | | NLR signaling |
| | | TLR Signaling |
| IL1B | interleukin 1, beta | Cytokine Signaling |
| | | Host-pathogen Interaction |
| | | Innate Immune System |
| | | Lymphocyte Activation |
| | | NF-kB Signaling |
| | | NLR signaling |
| | | Oxidative Stress |
| | | Th17 Differentiation |
| | | TNF Family Signaling |
| | | TLR Signaling |
| IL6 | interleukin 6 (interferon, beta 2) | Cytokine Signaling |
| | | Host-pathogen Interaction |
| | | Lymphocyte Activation |
| | | NLR signaling |
| | | Oxidative Stress |
| | | Th17 Differentiation |
| | | Th2 Differentiation |
| | | TNF Family Signaling |
| | | TLR Signaling |
| CD79A | CD79a molecule, immunoglobulin- | Adaptive Immune System |
| | associated alpha | B cell Receptor Signaling |
| | | Lymphocyte Activation |
| SOCS3 | suppressor of cytokine signaling 3 | Adaptive Immune System |
| | | Cytokine Signaling |
| | | Host-pathogen Interaction |
| | | MHC Class I Antigen Presentation |
| | | TNF Family Signaling |
| | | Type I Interferon Signaling |
| | | Type II Interferon Signaling |
| CXCL13 | chemokine (C-X-C motif) ligand 13 | Chemokine Signaling |
| | | Cytokine Signaling |
| CXCL1 | chemokine (C-X-C motif) ligand 1 | Chemokine Signaling |
| | (melanoma growth stimulating | Cytokine Signaling |
| | activity, alpha) | Host-pathogen Interaction |
| | | Innate Immune System |
| | | NLR signaling |
| | | TNF Family Signaling |
| PTGS2 | prostaglandin-endoperoxide | Cytokine Signaling |
| | synthase 2 (prostaglandin G/H | Host-pathogen Interaction |
| | synthase and cyclooxygenase) | Immunometabolism |
| | | NF-kB Signaling |
| | | Oxidative Stress |
| | | TNF Family Signaling |
| TNFRSF17 | tumor necrosis factor receptor | Cytokine Signaling |
| | superfamily, member 17 | |
| EGR1 | early growth response 1 | Cytokine Signaling |
| | | Host-pathogen Interaction |
| | | Lymphocyte Activation |
| | | Transcriptional Regulation |
| | | Type I Interferon Signaling |
| CXCL2 | chemokine (C-X-C motif) ligand 2 | Chemokine Signaling |
| | | Cytokine Signaling |
| | | Host-pathogen Interaction |
| | | NF-kB Signaling |
| | | NLR signaling |
| | | TNF Family Signaling |
| CCL20 | chemokine (C-C motif) ligand 20 | Chemokine Signaling |
| | | Cytokine Signaling |
| | | TNF Family Signaling |
| IL28A | interleukin 28A (interferon, lambda | Cytokine Signaling |
| | 2) | Lymphocyte Activation |
| CD19 | CD19 molecule | Adaptive Immune System |
| | | B cell Receptor Signaling |
| | | Complement System |
| | | Host-pathogen Interaction |
| | | Innate Immune System |
| LIF | leukemia inhibitory factor | Cytokine Signaling |
| | (cholinergic differentiation factor) | TNF Family Signaling |
| IL20 | interleukin 20 | Cytokine Signaling |
| XBP1 | X-box binding protein 1 | Host-pathogen Interaction |
| | | Lymphocyte Activation |
| | | Oxidative Stress |
| | | Transcriptional Regulation |
| BCL3 | B-cell CLL/lymphoma 3 | Lymphocyte Activation |
| | | TNF Family Signaling |
| | | Transcriptional Regulation |
| CXCR4 | chemokine (C-X-C motif) receptor | Chemokine Signaling |
| | 4 | Cytokine Signaling |
| | | Lymphocyte Trafficking |
| MIF | macrophage migration inhibitory | Cytokine Signaling |
| | factor (glycosylation-inhibiting | Hemostasis |
| | factor) | Innate Immune System |
| | | Lymphocyte Activation |
| CD79B | CD79b molecule, immunoglobulin- | Adaptive Immune System |
| | associated beta | B cell Receptor Signaling |
| | | Lymphocyte Activation |
| KLRG2 | killer cell lectin-like receptor | Lymphocyte Activation |
| | subfamily G, member 2 | |
| LTB4R | leukotriene B4 receptor | Immunometabolism |
| TNFRSF13C | tumor necrosis factor receptor | Cytokine Signaling |
| | superfamily, member 13C | Host-pathogen Interaction |
| | | Lymphocyte Activation |
| | | NF-kB Signaling |
| IRF3 | interferon regulatory factor 3 | Cytokine Signaling |
| | | Hemostasis |
| | | Host-pathogen Interaction |
| | | Innate Immune System |
| | | NLR signaling |
| | | TLR Signaling |
| | | Transcriptional Regulation |
| | | Type I Interferon Signaling |
| | | Type II Interferon Signaling |
| TNFAIP3 | tumor necrosis factor, alpha- | Host-pathogen Interaction |
| | induced protein 3 | Innate Immune System |
| | | Lymphocyte Activation |
| | | NF-kB Signaling |
| | | NLR signaling |
| | | Oxidative Stress |
| | | TNF Family Signaling |
| BCL2L11 | BCL2-like 11 (apoptosis facilitator) | Apoptosis |
| MAPKAPK2 | mitogen-activated protein kinase- | Cytokine Signaling |
| | activated protein kinase 2 | Immunometabolism |
| | | Innate Immune System |
| | | TLR Signaling |
| HLA-C | major histocompatibility complex, | Adaptive Immune System |
| | class I, C | Cell Adhesion |
| | | Cytokine Signaling |
| | | Host-pathogen Interaction |
| | | Innate Immune System |
| | | Lymphocyte Activation |
| | | MHC Class I Antigen Presentation |
| | | Phagocytosis and Degradation |
| | | Type I Interferon Signaling |
| | | Type II Interferon Signaling |
| IL1RAP | interleukin 1 receptor accessory | Cytokine Signaling |
| | protein | Th17 Differentiation |
| TRAF3 | TNF receptor-associated factor 3 | Cytokine Signaling |
| | | Host-pathogen Interaction |
| | | Innate Immune System |
| | | NF-kB Signaling |
| | | NLR signaling |
| | | TNF Family Signaling |
| | | TLR Signaling |
| CASP2 | caspase 2, apoptosis-related | Apoptosis |
| | cysteine peptidase | Innate Immune System |
| | | NLR signaling |
| MCL1 | myeloid cell leukemia sequence 1 | Apoptosis |
| | (BCL2-related) | Cytokine Signaling |
| | | Oxidative Stress |

It should be noted that the genes listed in Tables 1 and 2 may be associated with aspects of the host immune system. For example, one or more of the genes listed in these tables may encode factors which are immunomodulatory—that is they modulate one or more aspects of the innate or adaptive host immune response.

Based on the above and by way of a non-limiting example, the disclosure provides a method of downregulating the expression of the genes which encode IL8 (interleukin 8) and/or IL1B (interleukin 1, beta) and/or IL6 (interleukin 6), said method comprising administering a subject in need thereof, a quantity or amount of microwave energy as described herein. In one teaching, the subject may be suffering from a disease or condition caused or contributed to by the expression, for example aberrant expression of IL8 and/or IL1B and/or IL6.

The methods and uses described herein may additionally (or alternatively) be applied to modulating the expression of one or more of the genes listed in Table 3. Microwave energy may be used to upregulate the expression of one or more of the genes listed in Table 3.

TABLE 3

| Gene | Official full name | Participating pathways |
|---|---|---|
| THBS4 | thrombospondin 4 | PI3K |
| SFRP4 | secreted frizzled-related protein 4 | Wnt |
| RELN | reelin | PI3K |
| ETV1 | ets variant 1 | TXmisReg |
| TMPRSS2 | transmembrane protease, serine 2 | TXmisReg |
| MMP7 | matrix metallopeptidase 7 (matrilysin, uterine) | Wnt |
| PPARGC1A | peroxisome proliferator-activated receptor | ChromMod |
| | gamma, coactivator 1 alpha | |
| PLCB4 | phospholipase C, beta 4 | Wnt |
| PRKAR2B | protein kinase, cAMP-dependent, regulatory, type | CC+Apop |
| | II, beta | |
| AR | androgen receptor | Driver Gene |
| FGF2 | fibroblast growth factor 2 (basic) | MAPK, PI3K, RAS |
| GHR | growth hormone receptor | JAK-STAT, PI3K |
| PPARG | peroxisome proliferator-activated receptor gamma | TXmisReg |
| PLA2G4A | phospholipase A2, group IVA (cytosolic, calcium- | MAPK, RAS |
| | dependent) | |
| BCL2 | B-cell CLL/lymphoma 2 | Driver Gene, PI3K, CC+Apop |
| KIT | v-kit Hardy-Zuckerman 4 feline sarcoma viral | Driver Gene, PI3K, RAS |
| | oncogene homolog | |
| TGFB2 | transforming growth factor, beta 2 | TGF-B, MAPK, CC+Apop |
| NTRK2 | neurotrophic tyrosine kinase, receptor, type 2 | MAPK |
| ID4 | inhibitor of DNA binding 4, dominant negative | TGF-B |
| | helix-loop-helix protein | |
| PDGFD | platelet derived growth factor D | PI3K, RAS |
| VEGFC | vascular endothelial growth factor C | PI3K, RAS |
| KITLG | KIT ligand | PI3K, RAS |
| NGFR | nerve growth factor receptor | PI3K, RAS, TXmisReg |
| HDAC4 | histone deacetylase 4 | ChromMod |
| TSPAN7 | tetraspanin 7 | TXmisReg |
| LIFR | leukemia inhibitory factor receptor alpha | JAK-STAT |
| USP39 | ubiquitin specific peptidase 39 | HK |
| LIG4 | ligase IV, DNA, ATP-dependent | DNARepair |
| FGFR1 | fibroblast growth factor receptor 1 | MAPK, PI3K, RAS |
| B2M | beta-2-microglobulin | Driver Gene |
| ID2 | inhibitor of DNA binding 2, dominant negative | TXmisReg, TGF-B |
| | helix-loop-helix protein | |
| DDIT3 | DNA-damage-inducible transcript 3 | TXmisReg, MAPK |
| GPC4 | glypican 4 | Wnt |
| TGFBR2 | transforming growth factor, beta receptor II | TXmisReg, TGF-B, MAPK |
| | (70/80kDa) | |
| AKT3 | v-akt murine thymoma viral oncogene homolog 3 | MAPK, JAK-STAT, PI3K, RAS, |
| | | CC+Apop |
| ALKBH3 | alkB, alkylation repair homolog 3 (E. coli) | DNARepair |
| NOL7 | nucleolar protein 7, 27kDa | HK |
| MAPK1 | mitogen-activated protein kinase 1 | TGF-B, MAPK, PI3K, RAS |
| PRKACB | protein kinase, cAMP-dependent, catalytic, beta | Wnt, HH, MAPK, RAS, |
| | | CC+Apop |
| MAPK9 | mitogen-activated protein kinase 9 | MAPK, RAS |
| SKP1 | S-phase kinase-associated protein 1 | Wnt, TGF-B, CC+Apop |
| NF1 | neurofibromin 1 | Driver Gene, MAPK, RAS |
| SF3B1 | splicing factor 3b, subunit 1, 155kDa | Driver Gene |
| RPS27A | ribosomal protein 527a | DNARepair |

The methods and uses described herein may additionally (or alternatively) be applied to modulating the expression of one or more of the genes listed in Table 4. Microwave energy may be used to downregulate the expression of one or more of the genes listed in Table 4.

TABLE 4

| Gene | Official full name | Participating pathways |
|---|---|---|
| OSM | oncostatin M | JAK-STAT, PI3K |
| IL1B | interleukin 1, beta | MAPK, CC+Apop |
| GNG4 | guanine nucleotide binding protein (G protein), | PI3K, RAS |
| | gamma 4 | |
| FOS | FBJ murine osteosarcoma viral oncogene | MAPK |
| | homolog | |
| IL24 | interleukin 24 | JAK-STAT |
| IL6 | interleukin 6 (interferon, beta 2) | TXmisReg, JAK-STAT, PI3K |
| SOCS3 | suppressor of cytokine signaling 3 | JAK-STAT |
| NR4A1 | nuclear receptor subfamily 4, group A, member 1 | MAPK, PI3K |
| CD19 | CD19 molecule | PI3K |
| PAX5 | paired box 5 | Driver Gene, TXmisReg |
| DUSP2 | dual specificity phosphatase 2 | MAPK |
| LIF | leukemia inhibitory factor | JAK-STAT |
| BCL2A1 | BCL2-related protein A1 | TXmisReg |
| MYC | v-myc avian myelocytomatosis viral oncogene | Wnt, TXmisReg, TGF-B, |
| | homolog | MAPK, JAK-STAT, PI3K, |
| | | CC+Apop |
| EPHA2 | EPH receptor A2 | PI3K, RAS |
| FOSL1 | FOS-like antigen 1 | Wnt |
| CACNB3 | calcium channel, voltage-dependent, beta 3 | MAPK |
| | subunit | |
| ETS2 | v-ets avian erythroblastosis virus E26 oncogene | RAS |
| | homolog 2 | |
| HSPB1 | heat shock 27kDa protein 1 | MAPK |
| CDC7 | cell division cycle 7 | CC+Apop |
| COL27A1 | collagen, type XXVII, alpha 1 | PI3K |
| PIM1 | pim-1 oncogene | JAK-STAT |
| ID1 | inhibitor of DNA binding 1, dominant negative | TGF-B |
| | helix-loop-helix protein | |
| ALKBH2 | alkB, alkylation repair homolog 2 (E. coli) | DNARepair |
| TNFAIP3 | tumor necrosis factor, alpha-induced protein 3 | Driver Gene |
| CDKN2D | cyclin-dependent kinase inhibitor 2D (p19, inhibits | CC+Apop |
| | CDK4) | |
| MCM7 | minichromosome maintenance complex | CC+Apop |
| | component 7 | |
| VHL | von Hippel-Lindau tumor suppressor, E3 ubiquitin | Driver Gene |
| | protein ligase | |
| KRAS | Kirsten rat sarcoma viral oncogene homolog | Driver Gene, MAPK, PI3K, |
| | | RAS |
| PIK3R2 | phosphoinositide-3-kinase, regulatory subunit 2 | JAK-STAT, PI3K, RAS, |
| | (beta) | CC+Apop |
| HSP90B1 | heat shock protein 90kDa beta (Grp94), member 1 | MAPK |
| CIC | capicua transcriptional repressor | Driver Gene |
| PML | promyelocytic leukemia | TXmisReg |
| MMP3 | matrix metallopeptidase 3 (stromelysin 1, | TXmisReg |
| | progelatinase) | |
| TNFRSF10C | tumor necrosis factor receptor superfamily, | CC+Apop |
| | member 10c, decoy without an intracellular | |
| | domain | |
| IL8 | interleukin 8 | TXmisReg |

It should be noted that the genes listed in Tables 3 and 4 may be associated with the development, metastasis and/or progression of one or more types of cell proliferation and/or differentiation disorder, such as, for example, cancer. For example the expression, over expression or under expression of one or more of the genes listed in these tables may be classified as anti- or pro-cancer and may be linked (directly or indirectly) to disease progression. One of skill will appreciate that by appropriately modulating the expression of one or more of these genes, it may be possible to treat or prevent a cancer, including, for example a skin cancer.

For completeness, there follows a description of some of those pathways linked to one or more of the microwave modulated gene(s) described herein. One of skill may refer to these pathways as “key cancer pathways”.

Notch: Intercellular signaling mechanism essential for proper embryonic development. The Notch proteins are single-pass receptors and are transported to the plasma membrane as cleaved. Notch intracellular domain (NICD) translocates to the nucleus, where it forms a complex with the DNA binding protein CSL, displacing a histone deacetylase (HDAc)-co-repressor (CoR) complex from CSL. Notch signaling pathway can either act oncogenic or in a tumor-suppressive manner

APC (Wnt): Wnt proteins are secreted morphogens that are required for basic developmental processes, such as cell-fate specification, progenitor-cell proliferation and the control of asymmetric cell division, in many different species and organs.

HedgeHog: The Hedgehog (Hh) family of secreted signaling proteins plays a crucial role in development, regulating morphogenesis of a variety of tissues and organs. Hh signaling is also involved in control of stem cell proliferation in adult tissues and aberrant activation of the Hh pathway has been linked to multiple types of human cancer. Members of the Hh family bind to patched (ptc), thus releasing smoothened (smo) to transduce a signal.

Chromatin Modification: Members of this family of genes are involved or regulate processes associated with the alteration of DNA, protein, or sometimes RNA, in chromatin, which may result in changing the chromatin structure.

Transcriptional Regulation: A collection of pathways known to be transcriptionally misregulated in a variety of cancers.

DNA Damage: Control DNA repair is a multi-enzyme, multi-pathway system required to ensure the integrity of the cellular genome. DNA damage can arise spontaneously in the cellular milieu through chemical alteration of base nucleotides or as a consequence of errors during DNA replication.

TGF-B: The transforming growth factor-beta (TGF-beta) family members, which include TGF-betas, activins and bone morphogenetic proteins (BMPs), are structurally related secreted cytokines. A wide spectrum of cellular functions such as proliferation, apoptosis, differentiation and migration are regulated by TGF-beta family members.

MAPK: The mitogen-activated protein kinase (MAPK) cascade is a highly conserved module that is involved in various cellular functions, including cell proliferation, differentiation and migration. Abnormal MAPK signaling may lead to increased or uncontrolled cell proliferation and resistance to apoptosis.

JAK/STAT: The Janus kinase/signal transducers and activators of transcription (JAK/STAT) pathway is a pleiotropic cascade used to transduce a multitude of signals for development and homeostasis in animals. It is the principal signaling mechanism for a wide array of cytokines and growth factors which leads to activation of additional transcription factors.

PI3K: The phosphatidylinositol 3′-kinase(PI3K)-Akt signaling pathway regulates fundamental cellular functions such as transcription, translation, proliferation, growth, apoptosis, protein synthesis, metabolism cell cycle and survival.

RAS: The Ras proteins are GTPases that function as molecular switches for signaling pathways regulating cell proliferation, survival, growth, migration, differentiation or cytoskeletal dynamism.) Cell Cycle and Apoptosis: Mitotic cell cycle progression is accomplished through a reproducible sequence of events. Apoptosis is a genetically controlled mechanisms of cell death involved in the regulation of tissue homeostasis. The 2 major pathways of apoptosis are the extrinsic and the intrinsic both of which are found in the cytoplasm.

### DETAILED DESCRIPTION

The present invention will now be described with reference to the following figures which show:

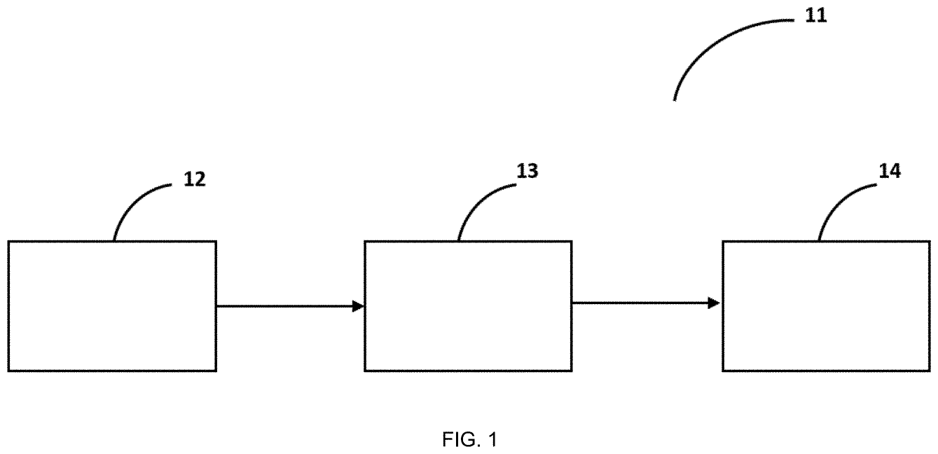

FIG. 1: a schematic illustration of a microwave treatment system, in accordance with embodiments.

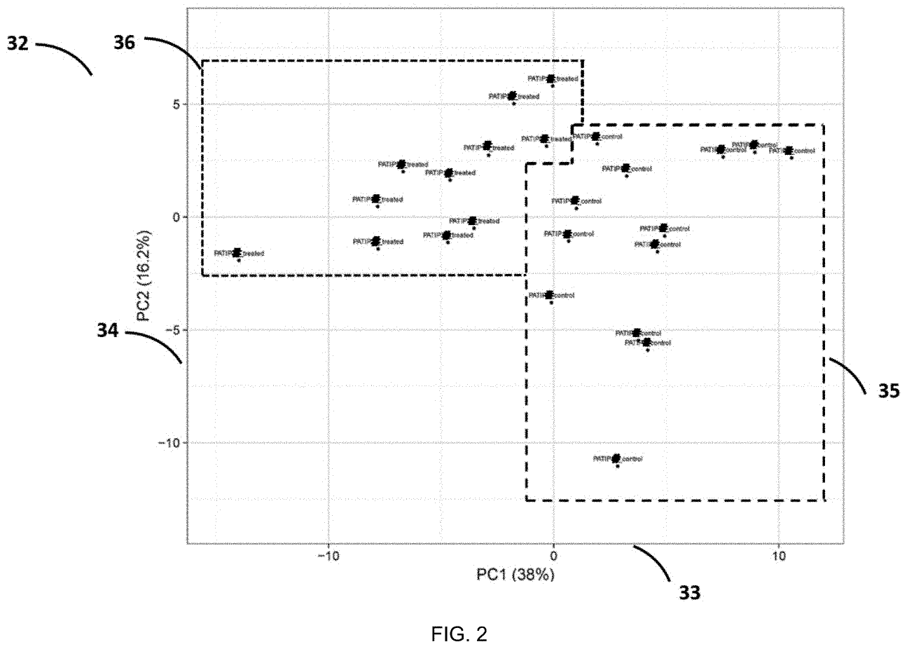

FIG. 2: PCA of significantly altered genes between microwave treated and untreated skin assessed on Immunology panel.

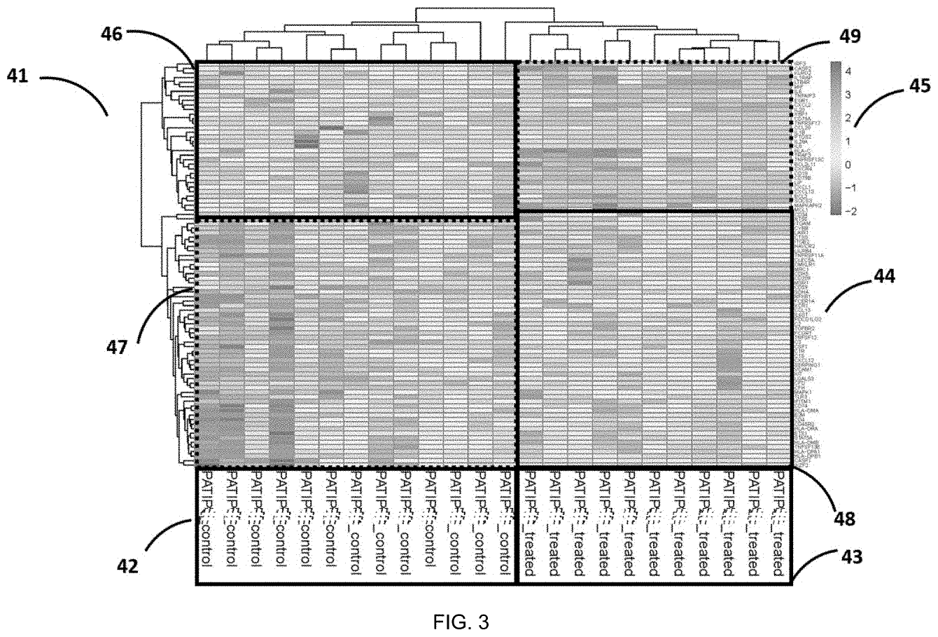

FIG. 3: a heatmap showing the significantly altered genes between microwave treated and untreated skin assessed on Immunology panel.

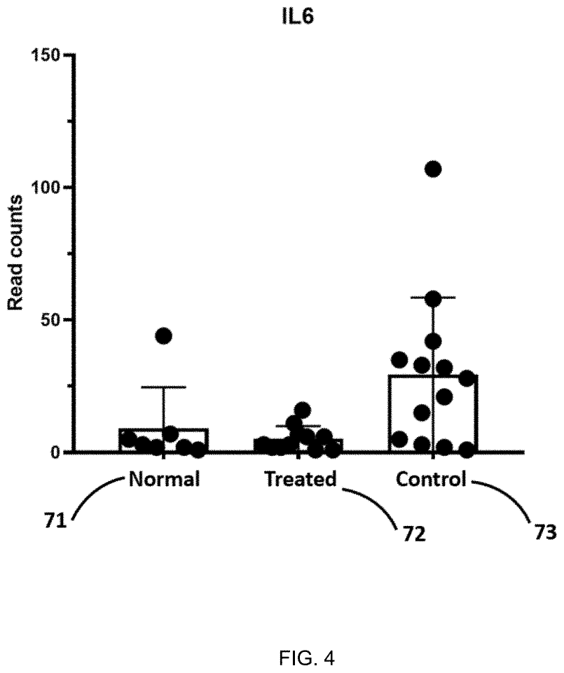

FIG. 4: Example of gene count in normal, microwave treated and diseased skin tissue on Immunology panel.

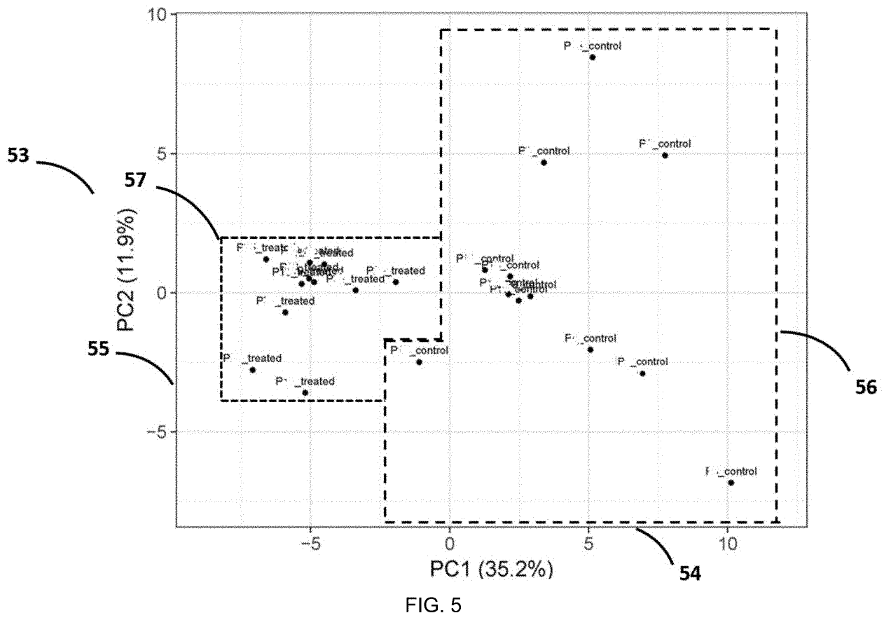

FIG. 5: PCA of significantly altered genes between microwave treated and untreated skin assessed on PanCancer panel.

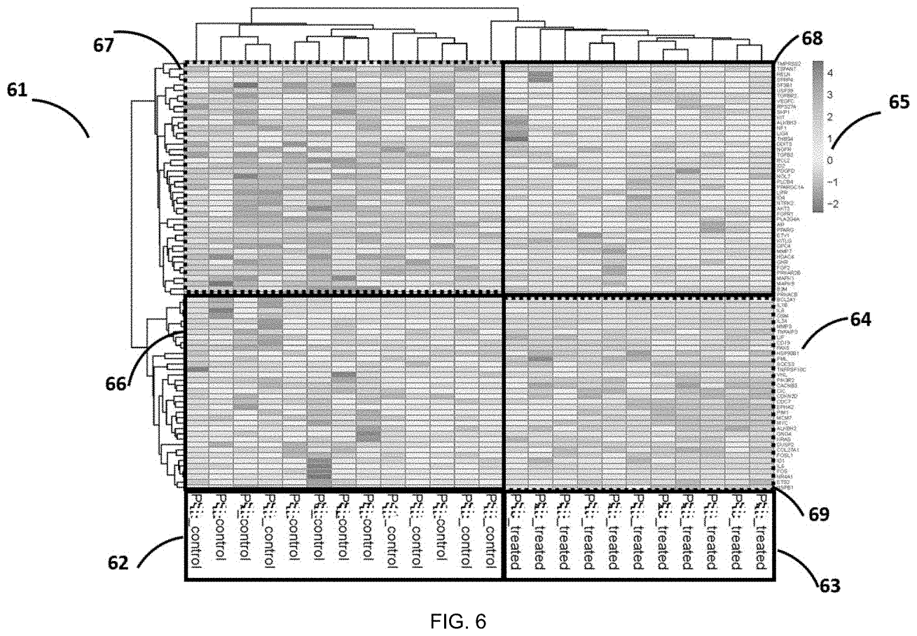

FIG. 6: a heatmap of significantly altered genes between microwave treated and untreated skin assessed on PanCancer panel.

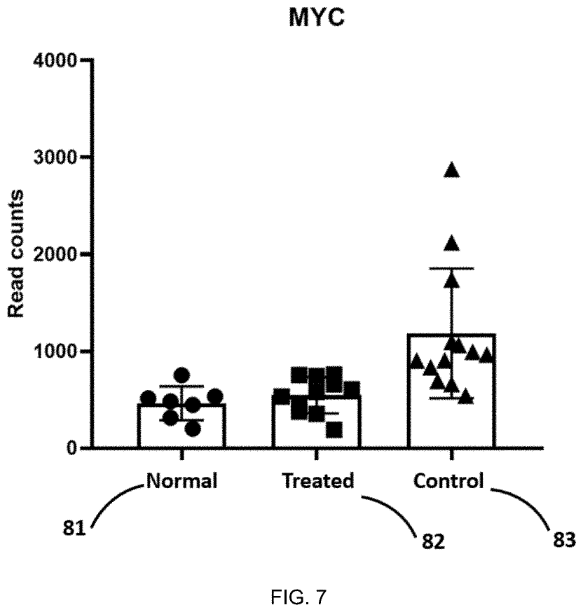

FIG. 7: Example of gene count in normal, microwave treated and diseased skin tissue on PanCancer panel.

### MICROWAVE ENERGY SYSTEM

A microwave radiation delivery system 11, in accordance with embodiments, for treating a biological tissue, is illustrated in FIG. 1. The system comprises a microwave generator 12 for generating microwave radiation, a flexible or rigid interconnecting cable 13 and a microwave applicator assembly (also called microwave antenna) 14 for delivering microwave radiation to a biological tissue. Other variations of this arrangement are possible including integrated versions. The microwave radiation delivery system may further comprise a controller (not shown) which is configured to select an operational frequency or range of frequencies to be supplied by the generator. A frequency of the microwave radiation supplied by the microwave generator 12 may be between 900 MHz and 30 GHz, for example about 915 MHz, about 2.45 GHz, about 5.8 GHz, about 8.0 GHz, or about 24.125 GHz. In some embodiments, the microwave radiation supplied is pulsed meaning that the energy is delivered in precise and brief doses lasting a number of seconds.

### Methods

A microwave system comprising a microwave generator; a controller configured to control the microwave generator to generate microwave energy having a selected operational frequency or range of frequencies; a microwave cable configured to deliver the microwave energy to a microwave antenna extending from or coupled to a distal end of the microwave cable; and a microwave antenna was used to apply microwave energy to skin tissue for example diseased skin tissue. This created thermal and non-thermal effects within the tissue and biopsies were taken for transcriptome studies.

The analysis of mRNA transcripts was performed using NanoString (NanoString Technologies Inc., Seattle, Wash. 98109 USA) nCounter gene expression system using, Immune pathway (nCounter Human Immunology V2 Panel, catalogue number: XT-CSO-HIM2-12) and Cancer pathway (nCounter PanCancer Pathways, catalogue number: XT-CSO-PATH1-12) to comprehend changes in the transcripts of number of genes (579 human genes in the Immunology V2 panel assay and 730 human genes in the PanCancer pathway assay) [20].

Gene expression analysis was performed using nSolver 4.0 (NanoString Technologies Inc., Seattle, Wash. 98109 USA) and open source Bioconductor DESeq2 in R studio (version 3.5.3). The results are presented in Tables 1-4 above and also 5-8 below.

Treated skin refers to microwave treated skin. Terms untreated skin/control/diseased skin are interchangeable.

TABLE 5
those genes found to be significantly
upregulated between microwave treated and
untreated skin assessed on Immunology panel.

| | Base | log2 Fold | | |
|---|---|---|---|---|
| Gene | Mean | Change | p-value | p-adj |
| CFH | 1026.38 | 1.49 | 0.0000 | 0.0000 |
| MSR1 | 157.37 | 1.44 | 0.0001 | 0.0015 |
| CXCL12 | 2043.39 | 1.24 | 0.0000 | 0.0000 |
| HLA-DPB1 | 2556.70 | 1.21 | 0.0000 | 0.0000 |
| MRC1 | 296.88 | 1.17 | 0.0000 | 0.0002 |
| FCER1A | 370.56 | 1.12 | 0.0003 | 0.0053 |
| C3 | 510.64 | 1.10 | 0.0000 | 0.0008 |
| VCAM1 | 117.58 | 1.00 | 0.0000 | 0.0000 |
| CFD | 577.43 | 0.96 | 0.0012 | 0.0132 |
| CCL13 | 291.62 | 0.87 | 0.0018 | 0.0184 |
| LGALS3 | 2115.07 | 0.85 | 0.0001 | 0.0019 |
| CDH5 | 139.47 | 0.83 | 0.0004 | 0.0062 |
| KIT | 129.87 | 0.82 | 0.0005 | 0.0072 |
| CD209 | 41.49 | 0.82 | 0.0028 | 0.0247 |
| LILRB4 | 65.57 | 0.79 | 0.0036 | 0.0298 |
| CLEC5A | 35.60 | 0.76 | 0.0045 | 0.0339 |
| HLA-DRA | 6024.43 | 0.76 | 0.0001 | 0.0024 |
| SERPING1 | 1521.07 | 0.75 | 0.0000 | 0.0003 |
| HLA-DPA1 | 2211.06 | 0.75 | 0.0001 | 0.0021 |
| CD4 | 220.85 | 0.73 | 0.0000 | 0.0002 |
| FCGRT | 940.18 | 0.73 | 0.0000 | 0.0003 |
| NT5E | 301.10 | 0.72 | 0.0058 | 0.0384 |
| C2 | 136.50 | 0.71 | 0.0000 | 0.0004 |
| CSF1 | 88.71 | 0.69 | 0.0000 | 0.0011 |
| HAVCR2 | 48.56 | 0.65 | 0.0035 | 0.0296 |
| TNFSF12 | 183.38 | 0.65 | 0.0000 | 0.0001 |
| LAIR1 | 158.32 | 0.65 | 0.0026 | 0.0245 |
| B2M | 22970.01 | 0.65 | 0.0008 | 0.0104 |
| CMKLR1 | 135.93 | 0.65 | 0.0006 | 0.0081 |
| PDCD1LG2 | 61.53 | 0.63 | 0.0000 | 0.0007 |
| CD45R0 | 303.52 | 0.63 | 0.0009 | 0.0115 |
| CD34 | 105.37 | 0.63 | 0.0047 | 0.0351 |
| XCR1 | 45.12 | 0.62 | 0.0030 | 0.0261 |
| CYBB | 338.71 | 0.61 | 0.0022 | 0.0220 |
| ITGAM | 79.52 | 0.60 | 0.0013 | 0.0145 |
| TGFBR2 | 634.57 | 0.55 | 0.0001 | 0.0024 |
| HLA-DMB | 393.95 | 0.53 | 0.0004 | 0.0066 |
| CTSS | 748.47 | 0.50 | 0.0056 | 0.0383 |
| IKZF2 | 58.96 | 0.50 | 0.0028 | 0.0247 |
| CD59 | 2503.76 | 0.49 | 0.0073 | 0.0443 |
| TNFRSF11A | 55.36 | 0.49 | 0.0027 | 0.0247 |
| TNFSF13B | 94.44 | 0.48 | 0.0068 | 0.0423 |
| TLR3 | 53.11 | 0.46 | 0.0049 | 0.0363 |
| STAT5A | 163.79 | 0.45 | 0.0002 | 0.0042 |
| IFITM1 | 1874.75 | 0.45 | 0.0068 | 0.0423 |
| NFKB1 | 151.67 | 0.44 | 0.0010 | 0.0118 |
| ITGB2 | 362.43 | 0.44 | 0.0078 | 0.0465 |
| C1S | 1402.07 | 0.43 | 0.0026 | 0.0245 |
| SDHA | 449.24 | 0.41 | 0.0016 | 0.0174 |
| ETS1 | 539.67 | 0.40 | 0.0032 | 0.0278 |
| CASP1 | 190.69 | 0.37 | 0.0057 | 0.0383 |
| C1R | 1843.39 | 0.37 | 0.0057 | 0.0383 |
| HLA-DMA | 489.96 | 0.36 | 0.0053 | 0.0383 |
| CD74 | 7154.25 | 0.35 | 0.0067 | 0.0422 |
| MAPK1 | 510.11 | 0.34 | 0.0055 | 0.0383 |
| IL6ST | 1035.06 | 0.32 | 0.0006 | 0.0084 |

TABLE 6
those genes significantly downregulated between
microwave treated and untreated skin assessed
on Immunology panel.

| | Base | log2 Fold | | |
|---|---|---|---|---|
| Gene | Mean | Change | p-value | p-adj |
| IL8 | 1285.14 | −3.51 | 0.0000 | 0.0008 |
| IL1B | 186.34 | −2.93 | 0.0000 | 0.0001 |
| IL6 | 20.73 | −2.33 | 0.0000 | 0.0006 |
| CD79A | 227.05 | −2.09 | 0.0003 | 0.0057 |
| SOCS3 | 405.62 | −2.06 | 0.0000 | 0.0000 |
| CXCL13 | 252.65 | −1.98 | 0.0000 | 0.0010 |
| CXCL1 | 405.92 | −1.98 | 0.0001 | 0.0025 |
| PTGS2 | 53.91 | −1.71 | 0.0001 | 0.0026 |
| TNFRSF17 | 61.18 | −1.42 | 0.0018 | 0.0183 |
| EGR1 | 148.70 | −1.41 | 0.0055 | 0.0383 |
| CXCL2 | 159.48 | −1.28 | 0.0002 | 0.0032 |
| CCL20 | 49.70 | −1.22 | 0.0039 | 0.0311 |
| IL28A | 6.95 | −1.17 | 0.0037 | 0.0308 |
| CD19 | 43.45 | −1.12 | 0.0004 | 0.0061 |
| LIF | 48.54 | −1.07 | 0.0001 | 0.0024 |
| IL20 | 22.40 | −0.99 | 0.0045 | 0.0339 |
| XBP1 | 993.22 | −0.94 | 0.0006 | 0.0081 |
| BCL3 | 353.32 | −0.91 | 0.0000 | 0.0004 |
| CXCR4 | 660.62 | −0.81 | 0.0000 | 0.0001 |
| MIF | 2064.06 | −0.74 | 0.0004 | 0.0065 |
| CD79B | 77.02 | −0.73 | 0.0071 | 0.0432 |
| KLRG2 | 39.89 | −0.73 | 0.0043 | 0.0334 |
| LTB4R | 597.46 | −0.64 | 0.0040 | 0.0319 |
| TNFRSF13C | 98.61 | −0.63 | 0.0054 | 0.0383 |
| IRF3 | 72.05 | −0.60 | 0.0008 | 0.0104 |
| TNFAIP3 | 313.05 | −0.59 | 0.0024 | 0.0231 |
| BCL2L11 | 71.16 | −0.55 | 0.0008 | 0.0104 |
| MAPKAPK2 | 278.49 | −0.51 | 0.0001 | 0.0027 |
| HLA-C | 3374.79 | −0.44 | 0.0042 | 0.0325 |
| IL1RAP | 223.61 | −0.43 | 0.0062 | 0.0403 |
| TRAF3 | 245.66 | −0.42 | 0.0026 | 0.0245 |
| CASP2 | 316.72 | −0.42 | 0.0076 | 0.0458 |
| MCL1 | 1935.98 | −0.40 | 0.0010 | 0.0118 |

TABLE 7
those genes that are significantly upregulated
genes between microwave treated and
untreated skin assessed on PanCancer panel;

| | Base | log2 Fold | | |
|---|---|---|---|---|
| Gene | Mean | Change | p-value | p-adj |
| THBS4 | 93.07 | 3.10 | 0.0000 | 0.0000 |
| SFRP4 | 22.13 | 1.88 | 0.0001 | 0.0030 |
| RELN | 20.60 | 1.65 | 0.0001 | 0.0023 |
| ETV1 | 34.49 | 1.55 | 0.0000 | 0.0000 |
| TMPRSS2 | 17.24 | 1.31 | 0.0016 | 0.0220 |
| MMP7 | 202.92 | 1.27 | 0.0007 | 0.0122 |
| PPARGC1A | 89.69 | 1.15 | 0.0000 | 0.0010 |
| PLCB4 | 47.93 | 1.13 | 0.0000 | 0.0001 |
| PRKAR2B | 141.05 | 1.11 | 0.0000 | 0.0004 |
| AR | 72.39 | 1.10 | 0.0002 | 0.0047 |
| FGF2 | 90.48 | 1.10 | 0.0000 | 0.0000 |
| GHR | 44.14 | 0.91 | 0.0000 | 0.0001 |
| PPARG | 61.65 | 0.87 | 0.0017 | 0.0220 |
| PLA2G4A | 58.95 | 0.79 | 0.0000 | 0.0008 |
| BCL2 | 103.81 | 0.77 | 0.0000 | 0.0000 |
| KIT | 72.69 | 0.76 | 0.0002 | 0.0050 |
| TGFB2 | 50.53 | 0.76 | 0.0001 | 0.0029 |
| NTRK2 | 334.85 | 0.73 | 0.0030 | 0.0307 |
| ID4 | 168.54 | 0.71 | 0.0030 | 0.0307 |
| PDGFD | 159.07 | 0.71 | 0.0050 | 0.0451 |
| VEGFC | 37.00 | 0.68 | 0.0027 | 0.0303 |
| KITLG | 309.93 | 0.67 | 0.0001 | 0.0036 |
| NGFR | 83.48 | 0.67 | 0.0007 | 0.0122 |
| HDAC4 | 172.01 | 0.66 | 0.0000 | 0.0000 |
| TSPAN7 | 119.23 | 0.65 | 0.0000 | 0.0013 |
| LIFR | 160.65 | 0.65 | 0.0007 | 0.0122 |
| USP39 | 190.37 | 0.60 | 0.0000 | 0.0000 |
| LIG4 | 107.93 | 0.58 | 0.0001 | 0.0024 |
| FGFR1 | 346.35 | 0.54 | 0.0017 | 0.0220 |
| B2M | 24036.71 | 0.54 | 0.0009 | 0.0141 |
| ID2 | 422.83 | 0.54 | 0.0028 | 0.0303 |
| DDIT3 | 142.36 | 0.53 | 0.0000 | 0.0007 |
| GPC4 | 53.80 | 0.53 | 0.0038 | 0.0369 |
| TGFBR2 | 787.94 | 0.52 | 0.0001 | 0.0020 |
| AKT3 | 183.97 | 0.49 | 0.0003 | 0.0057 |
| ALKBH3 | 130.76 | 0.42 | 0.0029 | 0.0305 |
| NOL7 | 409.12 | 0.35 | 0.0045 | 0.0415 |
| MAPK1 | 436.49 | 0.35 | 0.0025 | 0.0283 |
| PRKACB | 103.76 | 0.33 | 0.0058 | 0.0497 |
| MAPK9 | 192.79 | 0.32 | 0.0016 | 0.0220 |
| SKP1 | 2449.63 | 0.32 | 0.0003 | 0.0064 |
| NF1 | 276.22 | 0.29 | 0.0010 | 0.0160 |
| SF3B1 | 740.39 | 0.29 | 0.0018 | 0.0220 |
| RPS27A | 23365.03 | 0.22 | 0.0037 | 0.0364 |

!TABLE 8
those genes that are significantly
downregulated genes between microwave treated
and untreated skin assessed on PanCancer panel;

| | Base | log2 Fold | | |
|---|---|---|---|---|
| Gene | Mean | Change | p-value | p-adj |
| OSM | 88.91 | −4.09 | 0.0000 | 0.0000 |
| IL1B | 202.15 | −3.20 | 0.0000 | 0.0001 |
| GNG4 | 15.32 | −2.56 | 0.0000 | 0.0006 |
| FOS | 1526.49 | −2.54 | 0.0000 | 0.0002 |
| IL24 | 20.95 | −2.49 | 0.0000 | 0.0001 |
| IL6 | 19.49 | −2.22 | 0.0000 | 0.0007 |
| SOCS3 | 429.31 | −2.08 | 0.0000 | 0.0000 |
| NR4A1 | 94.69 | −2.00 | 0.0026 | 0.0293 |
| CD19 | 30.18 | −1.94 | 0.0003 | 0.0064 |
| PAX5 | 14.85 | −1.29 | 0.0020 | 0.0240 |
| DUSP2 | 59.50 | −1.16 | 0.0002 | 0.0052 |
| LIF | 45.57 | −1.16 | 0.0001 | 0.0027 |
| BCL2A1 | 53.99 | −1.06 | 0.0031 | 0.0311 |
| MYC | 889.67 | −1.03 | 0.0000 | 0.0001 |
| EPHA2 | 180.44 | −0.89 | 0.0000 | 0.0003 |
| FOSL1 | 106.32 | −0.82 | 0.0057 | 0.0495 |
| CACNB3 | 59.66 | −0.70 | 0.0001 | 0.0029 |
| ETS2 | 1508.69 | −0.66 | 0.0003 | 0.0062 |
| HSPB1 | 4060.38 | −0.66 | 0.0019 | 0.0233 |
| CDC7 | 120.29 | −0.66 | 0.0009 | 0.0147 |
| COL27A1 | 191.58 | −0.62 | 0.0002 | 0.0047 |
| PIM1 | 1315.32 | −0.59 | 0.0022 | 0.0256 |
| ID1 | 1020.68 | −0.54 | 0.0030 | 0.0307 |
| ALKBH2 | 67.57 | −0.53 | 0.0017 | 0.0220 |
| TNFAIP3 | 442.34 | −0.52 | 0.0021 | 0.0255 |
| CDKN2D | 116.20 | −0.51 | 0.0017 | 0.0220 |
| MCM7 | 662.05 | −0.48 | 0.0044 | 0.0407 |
| VHL | 478.69 | −0.47 | 0.0048 | 0.0434 |
| KRAS | 289.73 | −0.45 | 0.0028 | 0.0303 |
| PIK3R2 | 292.17 | −0.44 | 0.0016 | 0.0220 |
| HSP90B1 | 721.15 | −0.42 | 0.0007 | 0.0122 |
| CIC | 277.81 | −0.34 | 0.0005 | 0.0098 |
| PML | 617.66 | −0.32 | 0.0015 | 0.0220 |
| MMP3 | 186.51 | −0.23 | 0.0022 | 0.0261 |
| TNFRSF100 | 53.31 | −0.21 | 0.0043 | 0.0407 |
| IL8 | 1724.97 | −0.16 | 0.0000 | 0.0008 |

TABLE 9
shows significantly altered common genes between microwave treated
and untreated skin assessed on Immunology and PanCancer panel

| | Immunology Path | | | | Cancer Path | | | |
|---|---|---|---|---|---|---|---|---|
| | Base | log2 Fold | | | Base | log2 Fold | | |
| Gene | Mean | Change | pvalue | padj | Mean | Change | pvalue | padj |
| KIT | 129.87 | 0.82 | 0.0005 | 0.0072 | 72.69 | 0.76 | 0.0002 | 0.0050 |
| B2M | 22970.01 | 0.65 | 0.0008 | 0.0104 | 24036.71 | 0.54 | 0.0009 | 0.0141 |
| TGFBR2 | 634.57 | 0.55 | 0.0001 | 0.0024 | 787.94 | 0.52 | 0.0001 | 0.0020 |
| MAPK1 | 510.11 | 0.34 | 0.0055 | 0.0383 | 436.49 | 0.35 | 0.0025 | 0.0283 |
| IL8 | 1285.14 | −3.51 | 0.0000 | 0.0008 | 1724.97 | −0.16 | 0.0000 | 0.0008 |
| TNFAIP3 | 313.05 | −0.59 | 0.0024 | 0.0231 | 442.34 | −0.52 | 0.0021 | 0.0255 |
| LIF | 48.54 | −1.07 | 0.0001 | 0.0024 | 45.57 | −1.16 | 0.0001 | 0.0027 |
| CD19 | 43.45 | −1.12 | 0.0004 | 0.0061 | 30.18 | −1.94 | 0.0003 | 0.0064 |
| SOCS3 | 405.62 | −2.06 | 0.0000 | 0.0000 | 429.31 | −2.08 | 0.0000 | 0.0000 |
| IL6 | 20.73 | −2.33 | 0.0000 | 0.0006 | 19.49 | −2.22 | 0.0000 | 0.0007 |
| IL1B | 186.34 | −2.93 | 0.0000 | 0.0001 | 202.15 | −3.20 | 0.0000 | 0.0001 |

Results (see tables 1-9 above) comparing the level of genes expression in (microwave) untreated and (microwave) treated skin tissues (tested using Wald test) revealed:

89 genes in the Immunology V2 panel and 80 genes in the PanCancer pathway were significantly modulated (up and/or down regulated). All achieve significance at p<0.05.

### Immunology V2 Panel Results:

Total number of significantly altered genes between treated and untreated skin=89

Significantly upregulated genes (n=56) in the treated skin (see Tables 1 and 5):

B2M, C1R, C1S, C2, C3, CASP1, CCL13, CD209, CD34, CD4, CD59, CD74, CFH, CDH5, CFD, CXCL12, CLEC5A, CMKLR1, CSF1, CTSS, FCER1A, HLA-DPB1, MRC1, MSR1, CYBB, ETS1, FCGRT, HAVCR2, HLA-DMA, HLA-DMB, HLA-DPA1, HLA-DRA, IFITM1, IKZF2, IL6ST, ITGAM, ITGB2, KIT, LAIR1, LGALS3, LILRB4, MAPK1, NFKB1, NT5E, PDCD1LG2, CD45R0, SDHA, SERPING1, STAT5A, TGFBR2, TLR3, TNFRSF11A, TNFSF12, TNFSF13B, VCAM1, XCR1.

Significantly downregulated genes (n=33) in the treated skin (see Tables 2 and 6):

### BCL2L11, BCL3, CASP2, CCL20, CD19, CD79B, CXCR4, CD79A, CXCL1, HLA-C, CXCL13, IL1B, IL1RAP, IL20, IL28A, CXCL2, IL8, IRF3, KLRG2, EGR1, LTB4R, MAPKAPK2, MCL1, MIF, IL6, LIF, PTGS2, SOCS3, TNFAIP3, TNFRSF13C, TNFRSF17, TRAF3, XBP1.

In total forty-four genes were found to be significantly upregulated between treated and control skin. On the other hand, thirty-six genes were observed as significantly downregulated between treated and control skin.

### PanCancer Pathway Results:

Total number of significantly altered genes between treated and untreated skin=80 (see Tables 3 and 7)

Significantly upregulated genes (n=44) in the treated skin:

AKT3, ALKBH3, AR, B2M, BCL2, DDIT3, ETV1, FGF2, FGFR1, GHR, GPC4, HDAC4, ID2, ID4, KIT, KITLG, LIFR, LIG4, MAPK1, MAPK9, MMP7, NF1, NGFR, NOL7, NTRK2, PDGFD, PLA2G4A, PLCB4, PPARG, PPARGC1A, PRKACB, PRKAR2B, RELN, RPS27A, SF3B1, SFRP4, SKP1, TGFB2, TGFBR2, THBS4, TMPRSS2, TSPAN7, USP39, VEGFC.

Significantly downregulated genes (n=36) in the treated skin (see Tables 4 and 8):

### ALKBH2, BCL2A1, CACNB3, CD19, CDCl7, CDKN2D, CIC, COL27A1, DUSP2, EPHA2, ETS2, FOS, FOSL1, GNG4, HSP90B1, HSPB1, ID1, IL1B, IL24, IL6, IL8, KRAS, LIF, MCM7, MMP3, MYC, NR4A1, OSM, PAX5, PIK3R2, PIM1, PML, SOCS3, TNFAIP3, TNFRSF10C, VHL.

### Principal Component Analysis (PCA)

The analysis comprises principal component (PC) axes that elucidate distinguished distribution of control and treated skin samples. In FIG. 2, PCA 32 on the Immunology panel comprising first principal axis PC1 33 and second principal axis PC2 34 show variation of 38% and 16.2% respectively depicting 54.2% of variation in total between control and treated skin. Control skin data cells are distinctly clustered together in the region designated numeral 35 whereas treated data cells are prominently clustered together in the region designated 36.

Correspondingly, FIG. 5 illustrates PCA of the significantly transformed genes between treated and control skin on PanCancer panel. The first principal axis PC1 54 and second principal axes PC2 55 represent variation of 35.2% and 11.9% respectively totaling 47.1% variation between treated and control skin clustered distinctly in the regions designated 56 and 57 respectively.

### Heatmaps

Further, significantly altered genes between control and microwave treated skin are visualised based on hierarchical clustering and are demonstrated using heatmaps.

The columns/rows of the data matrix are re-ordered according to the hierarchical clustering result, putting similar observations close to each other.

Heatmaps in this document are shown on greyscale depicting blocks of ‘high’ and ‘low’ values on a scale.

In FIG. 3, the heatmap illustrates distinct distribution of significantly altered genes between untreated and microwave treated skin datasets on Immunology panel. Clustered gene dataset values are predominantly positive whereas gene values in the regions designated 47 and 49 are largely negative on the scale. Concurrently, FIG. 6 shows heatmap clustering of significantly altered genes on the PanCancer panel between untreated and microwave treated skin. Clustered gene dataset values between 66 and 68 are mainly positive whereas gene values in the regions designated 67 and 69 are largely negative on the scale.

### Gene Count Graphs

Example of gene count comparison between normal (healthy), treated (microwave treated) and control (diseased) skin tissue is depicted in FIG. 4 (Immunology panel) and FIG. 7 (PanCancer pathway).

In FIG. 4, IL6 (interleukin 6, interferon, beta 2) count in the tissue, for example a skin tissue, before 73 and after microwave treatment 72 is shown and is compared with a normal tissue 71 for example healthy skin. IL6 participates in numerous important immunomodulatory pathways such as Cytokine Signaling, Host-pathogen Interaction, Lymphocyte Activation, NLR signalling, Oxidative Stress, Th17 Differentiation, Th2 Differentiation, TNF Family Signaling and TLR Signaling. Microwave treated skin has shown to restore the abnormally upregulated expression of the IL6 gene from the diseased skin and downregulate it to be equivalent to the normal healthy skin.

Similarly, in FIG. 7, gene count of MYC (v-myc avian myelocytomatosis viral oncogene homolog) a proto-oncogene that plays a vital role in cell cycle progression, apoptosis and cellular transformation is shown. MYC also participates in key cancer pathways such as Wnt, TXmisReg, TGF-B, MAPK, JAK-STAT and PI3K. MYC read count is aberrantly upregulated in the diseased skin 83 as compared to the normal skin 81. Upon microwave treatment, the gene count is downregulated and restored 82.

### Commonly Affected Genes

Furthermore, as shown in Table 9, eleven common genes in Immunology and PanCancer panels were found to be significantly altered between microwave treated and control skin.

Common Significantly Altered Genes in PanCancer and Immunology V2 Panel (See Table 9: n=11):

Significantly upregulated (n=4): TGFBR2, KIT B2M, MAPK1

Significantly downregulated (n=7): SOCS3, IL1B, IL6, IL8, LIF, CD19, TNFAIP3

The significantly transformed genes in the Human Immunology V2 Panel participate in and modulate various cellular pathways and contribute to vital aspects of the immune system such as adaptive immune system, apoptosis, autophagy, B cell receptor signalling, cell adhesion, chemokine signalling, complement system, cytokine signalling, haemostasis, host-pathogen interaction, immunometabolism, inflammasomes, innate immune system, lymphocyte activation, lymphocyte trafficking, MHC Class I Antigen Presentation, MHC Class II Antigen Presentation, NF-kB signalling, NLR signalling, oxidative stress, phagocytosis and degradation, T cell receptor signalling, TGF-b signalling, Th1 differentiation, Th17 differentiation, Th2 differentiation, TNF family signalling, TLR signalling, Transcriptional regulation, Treg differentiation, Type I Interferon signalling and Type II Interferon signalling.

Significantly dysregulated genes on the PanCancer pathway participate in several key cancer pathways such as Notch, APC (Wnt), Hedgehog, Chromatin Modification, Transcriptional Regulation, DNA Damage Control, TGF-B, MAPK, JAK-STAT, PI3K, RAS, cell cycle and apoptosis [20].

### REFERENCES

[1] X. L. Wang, H. W. Wang, K. Yuan, F. L. Li and Z. Huang, “Combination of photodynamic therapy and immunomodulation for skin diseases-update of clinical aspects.,” Photochemical& Photobiological Sciences, vol. 10, no. 5, p. 704., 2011.
[2] U. R. Hengge and T. Ruzicka, “Topical Immunomodulation in Dermatology: Potential of Toll-like Receptor Agonists,” Dermatologic Surgery, vol. 30, no. 8, pp. 1101-1112, 2004.
[3] J. W. Hadden, “Immunopharmacology,” JAMA, vol. 268, no. 20, pp. 2964-69, 1992.
[4] S. Reitamo, A. Remitz, H. Kyllönen and J. Saarikko, “Topical noncorticosteroid immunomodulation in the treatment of atopic dermatitis.,” Am J Clin Dermatol., vol. 3, no. 6, pp. 381-8., 2002.
[5] S. H. Ibbotson, “Topical 5-aminolaevulinic acid photodynamic therapy for the treatment of skin conditions other than non-melanoma skin cancer.,” British Journal of Dermatology, vol. 146, no. 2, pp. 178-188, 2002.
[6] J. Feehan, S. Burrows, L. Cornelius, A. M. Cook, K. Mikkelsen, V. Apostolopoulos, M. Husaric and D. Kiatos, “Therapeutic applications of polarized light: Tissue healing and immunomodulatory effects.,” Maturitas, vol. 116, pp. 11-17, 2018.
[7] Q. Huang, J. Hu, F. Lohr, L. Zhang, R. Braun, J. Lanzen, J. Little, M. Dewhirst and C. Li, “Heat-induced gene expression as a novel targeted cancer gene therapy strategy,” Cancer Res, vol. 60, no. 13, pp. 3435-9, 2000.
[8] X. Li, X. Gao, L. Jin, Y. Wang, U. Hong, X. McHepange, Y. Wang, U. Jiang, J. Wei and H.-D. Chen, “Local hyperthermia could induce migrational maturation of Langerhans cells in condyloma acuminatum,” J Dermatol Sci, vol. 54, no. 2, pp. 121-3, 2009.
[9] J. Q. Del-Rosso, “Topical Imiquimod Therapy for Actinic Keratosis. Is Long-Term Clearance a Realistic Benefit?,” J Clin Aesthet Dermatol., vol. 1, no. 3, pp. 44-47, 2008.
[10] S. Adams, L. Kozhaya, F. Martiniuk, T. Meng, L. Chiriboga, L. Liebes, T. Hochman, N. Shuman, D. Axelrod, J. Speyer, Y. Novik, A. Tiersten, J. Goldberg, C. Formenti, N. Bhardwaj, D. Unutmaz and S. Demaria, “Topical TLR7 agonist imiquimod can induce immune-mediated rejection of skin metastases in patients with breast cancer.,” Clin Cancer Res., vol. 18(24), no. 24, pp. 6748-57., 2012.
[11] A. H. GmbH, “DICLOFENAC SODIUM 3% GEL,” 2019. [Online]. Available: https-J/www.drugs.com/uk/diclofenac-sodium-3-gel-leaflet.html. [Accessed 17 Oct. 2019].
[12] K. Thai, P. Fergin, M. Freeman, C. Vinciullo, D. Francis, L. Spelman, D. Murrell, C. Anderson, W. Weightman, C. Reid, A. Watson and P. Foley, “A prospective study of the use of cryosurgery for the treatment of actinic keratoses.,” Int J Dermatol., vol. 43(9), no. 9, pp. 687-92., 2004 September
[13] A. Dodds, A. Chia and S. Shumack, “Actinic Keratosis: Rationale and Management,” Dermatol Ther (Heidelb), pp. 11-31, 2014.
[14] N. Krawtchenko, J. Roewert-Huber, M. Ulrich, I. Mann, W. Sterry and E. Stockfleth, “A randomised study of topical 5% imiquimod vs. topical 5-fluorouracil vs. cryosurgery in immunocompetent patients with actinic keratoses: a comparison of clinical and histological outcomes including 1-year follow-up.,” Br J Dermatol., vol. 157, pp. Suppl 2:34-40., 2007.
[15] S. Corr, N. Jared, C. Steven and A. Sikora, “Radiofrequency Field Hyperthermia And Solid Tumor Immunomodulation”. WIPO Patent WO 2018/071837 A1, 19 Apr. 2018.
[16] J. Tyler, A. Jaeger, S. Santagata, L. Whitesell and S. Lindquist, “Combination Treatments Of Hsp90 Inhibitors For Enhancing Tumor Immunogenicity And Methods Of Use Thereof”. WIPO Patent WO 2019/232533 A1, 5 Dec. 2019.
[17] F. Barrat, “Methods Of Treating Cancer, Infectious Disease, And Autoimmune Disease Using Cxc Chemokines”. US Patent US 2018/0193382 A1, 12 Jul. 2018.
[18] S. Ferree, J. Cowens, C. Jorgensen, T. Nielsen and B. Ejlertsen, “Methods Of Treating Breast Cancer With Gemcitabine Therapy”. US Patent US 2014/0037620 A1, 622014.
[19] L. Radvanyi, J. Chen and H. Patrick, “Biomarkers And Targets For Cancer Immunotherapy”. WIPO Patent WO 2016/073748 A1, 12 May 2016.
[20] F.-C. Tsai, G.-J. Chang, Y.-J. Hsu, Y.-M. Lin, Y.-S. Lee, W.-J. Chen, C.-T. Kuo and Y.-H. Yeh, “Proinflammatory gene expression in patients undergoing mitral valve surgery and maze ablation for atrial fibrillation,” The Journal of Thoracic and Cardiovascular Surgery, vol. 151, no. 6, pp. 1673-1682.e5, 2015.
[21] “NanoString Technologies,” Seattle, Wash., United States, 2019. [Online]. Available: https-J/www.nanostring.com. [Accessed 17 Oct. 2019].

What is claimed is:

1. A method of modulating the expression of one or more genes, said method comprising administering microwave energy to a subject in need thereof.

2. The method of claim 1, wherein the microwave energy administered at a frequency of between about 900 MHz and about 200 GHz.

3. The method of claim 1, wherein the microwave energy is administered at about 915 MHz, at about 2.45 GHz, at about 5.8 GHz, at about 8.0 GHz, or at about 24.125 GHz.

4. The method of claim 1, wherein the microwave energy is administered at an input power of 0.5 W to 40 W.

5. The method of claim 1, wherein the microwave energy is administered for a duration of anywhere between about 0.1 s to 20 s.

6. The method of claim 1, wherein the microwave energy is administered at 5 W for 3s, 4 W for 3s or 3 W for 3s.

7. The method of claim 1, wherein the microwave energy is administered as a series of pulsed doses.

8. The method of claim 7, wherein each pulse dose in the series of pulses is separated from another pulsed dose in the series by a time gap of anywhere between about 1 s to about 60 s

9. The method of claim 1, wherein the microwave energy is administered as 3 doses with a 20 s-time gap between each administered dose.

10. The method of claim 1, wherein the administered microwave energy is non-ablative.

11. The method of claim 10, wherein the non-ablative microwave energy does not cause direct tissue or skin damage.

12. The method of claim 1, wherein one or more of the gene to be modulated encode or provide factors associated with the host immune system.

13. The method of claim 1, wherein one or more of the gene to be modulated encode or provide immunomodulatory factors.

14. The method of claim 1, wherein one or more of the genes to be modulated are oncogenes.

15. The method of claim 1, wherein the one or more genes are presented in Tables 1 or 3.

16. The method of claim 1, wherein the one or more genes are presented in Tables 2 or 4.

17. A method of treating or preventing a skin condition by modulating the expression of one or more genes, said method comprising administering a subject suffering from, or predisposed to, the skin condition, microwave energy.

18. The method of claim 17, wherein the one or more genes are associated with the skin condition to be treated or prevented.

19. The method of claim 17, wherein the gene or genes to be modulated may be directly or indirectly associated with a disease or condition affecting the skin and/or wherein one or more of the genes may be involved or linked with/to one or more pathways or mechanisms associated with a disease or condition of the skin.

20. The method of claim 17, wherein the microwave energy administered at a frequency of between about 900 MHz and about 200 GHz.

21. The method of claim 17, wherein the microwave energy is administered at about 915 MHz, at about 2.45 GHz, at about 5.8 GHz, at about 8.0 GHz, or at about 24.125 GHz.

22. The method of claim 17, wherein the microwave energy is administered at an input power of 0.5 W to 40 W.

23. The method of claim 17, wherein the microwave energy is administered for a duration of anywhere between about 0.1 s to 20 s.

24. The method of claim 17, wherein the microwave energy is administered at 5 W for 3s, 4 W for 3s or 3 W for 3s.

25. The method of claim 17, wherein the microwave energy is administered as a series of pulsed doses.

26. The method of claim 25, wherein each pulse dose in the series of pulses is separated from another pulsed dose in the series by a time gap of anywhere between about 1 s to about 60 s

27. The method of claim 17, wherein the microwave energy is administered as 3 doses with a 20 s-time gap between each administered dose.

28. The method of claim 17, wherein the administered microwave energy is non-ablative.

29. The method of claim 28, wherein the non-ablative microwave energy does not cause direct tissue or skin damage.

30. The method of claim 17, wherein the expression of one or more of the genes is/are either (i) downregulated, inhibited or reduced or (ii) upregulated, induced, promoted or stimulated.

31. The method of claim 17, wherein the microwave energy is administered to a diseased tissue.

32. The method of claim 17, wherein the microwave energy is administered to a tissue exhibiting the signs or symptoms characteristic of one or more diseases.

33. The method of claim 17, wherein the microwave energy is administered to the skin and/or diseased skin.

34. The method of claim 17, wherein the microwave energy is administered to:
(i) aging skin; or
(ii) skin which exhibits solar damage; or
(iii) skin with one or more scars, erosion and/or lesions.

35. The method of claim 17, wherein the disease to be treated or prevented is a wart, eczema, psoriasis, acne, cherry angioma, hidradenitis suppurativa, rosacea, ichthyosis, keloid scars, seborrheic dermatitis, seborrheic keratosis, seborrheic hyperplasia, Sebaceous hyperplasia, basal cell carcinoma, actinic keratosis, syringoma, squamous cell carcinoma, nevus, lentigo maligna, Melasma, melanoma, milia, molluscum contagiosum, cervical intraepithelial neoplasia, vaginal intraepithelial neoplasia, vulvar intraepithelial neoplasia, Bowen's disease and/or erythroplasia of queyrat.

36. The method of claim 17, wherein the disease to be treated or prevented is gastric epithelial dysplasia, cardiovascular lesions, conditions involving oral cavity such as epithelial dysplasia, leukoplakia, hairy leukoplakia, erythroplakia, erythroleukoplakia, lichen planus, xerostomia, mucositis, pyogenic granuloma, angioma, nicotinic stomatitis, actinic cheilitis, keratoacantoma, hyperkeratosis, candidosis, erythema migrans and/or a canker sore.

37. A method of treating a disease or condition associated with the downregulation or inhibition of a particular gene, said method comprising administering a subject in need thereof microwave energy to upregulate the gene thereby restoring the level of expression, activity and/or function of the gene to treat or prevent the disease or condition.

38. The method of claim 37, wherein the disease or condition is a disease or condition of the skin.

39. A method of treating a disease or condition associated with the upregulation of a particular gene, said method comprising administering a subject in need thereof microwave energy to downregulate the gene thereby restoring the level of expression, activity and/or function of the gene to treat or prevent the disease or condition.

40. The method of claim 39, wherein the disease or condition is a disease or condition of the skin.
